(19) United States
(12) Patent Application Publication
(10) Pub. No.: US 2008/0008742 A1
(43) Pub. Date: Jan. 10, 2008

(54) Chewy products and methods for making the same

(75) Inventor: Subraman Rao Cherukuri, Vienna, VA (US)

Correspondence Address: BUCHANAN, INGERSOLL & ROONEY PC, POST OFFICE BOX 1404, ALEXANDRIA, VA, 22313-1404, US

(73) Assignee: CAPRICORN PHARMA, INC., Frederick, MD (US)

(21) Appl. No.: 11/819,902

(22) Filed: Jun. 29, 2007

Related U.S. Application Data
(60) Provisional application No. 60/806,241, filed on Jun. 29, 2006.

(51) Int. Cl. A61K 47/00; A61K 9/20

(52) U.S. Cl. 424/439; 424/464

(57) ABSTRACT

Methods for producing chewable emulsion compositions and compositions produced from such methods are disclosed. The emulsion base may be prepared by contacting an aqueous phase and a nonaqueous phase in the presence of an emulsifier. The aqueous phase is prepared under conditions that permit no substantial moisture loss and reduced viscosity. The methods permit preparing chewable compositions for delivering confectionary items, nutriceuticals, vitamins, minerals and therapeutically active agents. The compositions may be sugar-free or sugar-containing. The active agent may be added to the emulsion base at a lower temperature compared to the temperature at which either the aqueous or nonaqueous phase is formed. The compositions may be further coated with taste-masking materials, enteric coating materials, or slow-release materials. The compositions may be formed into compressible tablets, beads, granules, or otherwise generally known dosage forms.

### CROSS-REFERENCE TO RELATED APPLICATIONS

This application claims priority under 35 U.S.C. § 119 to U.S. Provisional Application No. 60/806,241 entitled “Chewy Products and Methods for Making the Same” and filed on Jun. 29, 2006, the entire content of which is hereby incorporated by reference in its entirety for all purposes.

### FIELD OF THE INVENTION

The invention relates to confectionary and pharmaceutical fields. In particular, the invention relates to chewy products and methods for preparing the same in the confectionary and pharmaceutical fields.

### BACKGROUND OF THE INVENTION

Pharmaceutical compositions may be produced in a variety of dosage forms, depending upon the desired route of administration of the therapeutic material. For example, oral dosage forms may include such solid compositions as tablets, emulsions, and suspensions. The particular dosage form utilized will depend on such factors as the solubility and chemical reactivity of the pharmaceutical active. Further, the dosage form may be selected to optimize delivery of the pharmaceutical active and/or consumer acceptability of the composition.

Tablet compositions offer many advantages, including ease of product handling, chemical and physical stability, portability (in particular, allowing ready availability to the consumer when needed), aesthetic acceptability and dosage precision, i.e., ensuring consistent and accurate dosages of the pharmaceutical active. However, liquid formulations may also offer advantages in the treatment of certain disorders, such as disorders of the upper gastrointestinal tract, where delivery of an active material dissolved or dispersed in a liquid ensures rapid and complete delivery to the afflicted area. In an effort to obtain the therapeutic advantages associated with liquid formulations as well as the broad advantages associated with solids, many chewable tablet formulations have been developed.

Another product often used to deliver active agents to patients is emulsions. An emulsion is a dispersed system containing at least two immiscible liquids. The majority of conventional emulsions in pharmaceutical use have dispersed particles ranging in diameter from 0.1 to 100 microns. Emulsions are often thermodynamically unstable as a result of the excess free energy associated with the surface of the particles. The dispersed particles, therefore, strive to come together and reduce the surface area. In addition to this flocculation effect, the dispersed particles can coalesce, or fuse. This effect can result in the eventual destruction of the emulsion. In order to minimize this effect, a third component, the emulsifying agent, is often added to the system to improve stability. The choice of emulsifying agent is critical to the preparation of an emulsion possessing optimum stability.

Mostly, one of the two emulsified components is aqueous, while the other component is nonaqueous, for example, fatty substances such as oils. Many aqueous emulsions are prepared at elevated temperatures, i.e., at temperatures greater than 175° F. The elevated temperature aids in the dispersal of the non-aqueous component into the aqueous component by the emulsifying agent. However, if the high temperature emulsion is cooled in order to solidify the mixture, separation of the fatty substance, or “oiling off” may be observed in the final product if the levels of the fatty substance are too high or if the fatty substance is not added at the proper temperature.

A further disadvantage of high temperature emulsions is the detrimental effect of the high temperature on the efficacy and stability of active agents added to the emulsion. Many active agents, whether the active agent is a flavor, pharmaceutical, or nutriceutical, are not stable at high temperatures. Thus, if the active agents are added at the high temperatures, the active agents break down, resulting in uneven dosing (or doses that contain no active agents) and waste of the active agents.

Furthermore, many pharmaceutical and nutritional active agents have a bitter taste, so a flavor-enhancer or taste-masking agent is often added to oral doses containing the pharmaceutical or nutriceutical. However, the addition of a flavor-enhancer or taste-masking agent results in extra care needed to ensure that neither the pharmaceutical or nutriceutical, nor the flavor enhancer, is destroyed during processing of the high temperature emulsion prior to cooling to a solid base.

Examples of confectionary delivery systems known in the art include U.S. Pat. No. 4,582,709; U.S. Pat. No. 4,778,676; U.S. Pat. No. 5,637,313; Canadian Patent Application No. 2,165,838; U.S. Pat. No. 4,971,798; U.S. Pat. No. 4,963,359; European Patent No. 0 273 001; U.S. Pat. No. 5,637,313; U.S. Pat. No. 5,476,678; U.S. Pat. No. 4,778,676; WO 03/026438; U.S. Pat. No. 6,517,886, U.S. Pat. No. 5,637,313, U.S. Pat. No. 4,582,709, and U.S. Pat. No. 6,248,363.

However, the confectionary delivery systems known in the art provide compositions and processes that are often inefficient, of high-temperature, or not suitable for actives that are temperature sensitive. In some cases, the product obtained from such processes may not of desirable texture and mouth-feel, because of a considerable degree of aeration.

### SUMMARY OF THE INVENTION

Applicant has developed novel methods for preparing chewable compositions. In one aspect, the method comprises:

a) preparing a nonaqueous phase by applying thermal energy, radiation, or mechanical force to a solid or semisolid nonaqueous composition;

b) preparing an aqueous liquid phase by applying thermal energy, radiation, or mechanical force to an aqueous composition without substantial moisture loss such that the aqueous phase has reduced viscosity compared to said aqueous composition;

c) contacting said nonaqueous phase with said aqueous phase to obtain an emulsion; and

d) forming a chewable base material for delivering an active agent.

In one aspect, optional ingredients such as fillers, colorants, humectants, preservatives, taste-masking agents, sweeteners, stabilizers, lubricants may be incorporated into the chewable base material.

In a further aspect, an active agent is incorporated into the chewable base material at a temperature below that of either forming the nonaqueous phase (step (a)) or forming aqueous phase (step (b)).

Chewy compositions prepared according the processes herein are also disclosed.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

Methods for making and using chewy compositions are disclosed. In one aspect, the method comprises:

a) preparing a nonaqueous phase by applying thermal energy, radiation, or mechanical force to a solid or semisolid nonaqueous composition;

b) preparing an aqueous liquid phase by applying thermal energy, radiation, or mechanical force to an aqueous composition without substantial moisture loss such that the aqueous phase has reduced viscosity compared to said aqueous composition;

c) contacting said nonaqueous phase with said aqueous phase to obtain an emulsion; and

d) forming a chewable base material for delivering an active agent.

In describing and claiming the present invention, the following terminology will be used in accordance with the definitions set forth below.

The singular forms “a,” “an,” and, “the” include plural referents unless the context clearly dictates otherwise. Thus, for example, reference to “a drug” includes reference to one or more of such drugs, and reference to “an excipient” includes reference to one or more of such excipients.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients.

The phrase “chewy composition” or “chewable composition” as used herein represents a composition that is preferably intended to be masticated in the mouth of a mammal. When such mastication occurs, the composition of the present invention may break up or disintegrate or becomes erodible such that the composition loses a portion of its original weight or shape or both over time. In some aspects, these compositions are distinct from what are generally known in the art as chewing gums, nougats (which are highly aerated) or hard candy.

As used herein, “active agent,” “bioactive agent,” “pharmaceutically active agent,” and “pharmaceutical,” may be used interchangeably to refer to an agent or substance that has measurable specified or selected physiologic activity when administered to a subject in a significant or effective amount. It is to be understood that the term “drug” is expressly encompassed by the present definition as many drugs and prodrugs are known to have specific physiologic activities. These terms of art are well-known in the pharmaceutical, and medicinal arts. Non-limiting examples of “active agents” which can be used in the present invention include dietary fiber, stannol esters, therapeutically active substances, vitamins, minerals, antacids, cough and cold medications, analgesics, cardiovasular medications, anti-smoking, psycho-therapeutics, antibiotics, and mixtures thereof.

As used herein, “subject” refers to a mammal that may benefit from the administration of a drug composition or method of this invention. Examples of subjects include humans, and may also include other animals such as horses, pigs, cattle, dogs, cats, rabbits, and aquatic mammals.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, “pharmaceutically acceptable carrier” and “carrier” may be used interchangeably, and refer to any inert and pharmaceutically acceptable material that has substantially no biological activity, and makes up a substantial part of the formulation.

The term “admixed” means that the drug and/or other ingredients can be dissolved, dispersed, or suspended in the carrier. In some cases, the drug may be uniformly admixed in the carrier.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, the term “sugar-free” and “sugarless” refer to the compositions that are substantially free (i.e. contain less than about 10% by weight, and preferably less than 0.1% by weight) of fermentable sugars such as glucose and corn syrups, which help promote tooth decay. The terms “sugar-free” and “sugarless” are meant to be used interchangeably here.

The expression “soft chew” includes items which are solid at room temperature, and those items which may be considered semi-solid or soft chew at room temperature. In general, the term “soft chew” is meant to cover those items that are not liquid or gas at room temperature. Further, these compositions are distinct from chewing gums, nougats (which are highly aerated) and hard candies.

The expression “emulsifier system” means a component that comprises emulsifiers, fats and mixtures thereof.

As used herein, “remote location” means a location that is separate from the location in which the composition is prepared according to the present inventive process.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

In forming compositions according to the invention, a nonaqueous phase and an aqueous liquid phase are prepared. The nonaqueous phase is prepared by applying thermal energy (such as heat), radiation (such us microwave, or laser) or mechanical force (such as high shear or low shear, centrifugation, vortex mixing, etc) to a solid or semisolid nonaqueous composition.

In some aspects, the nonaqueous phase is prepared at a temperature ranging from about 55° C. to about 120° C. Preferably, the nonaqueous phase is prepared at a temperature ranging from about 55° C. to about 100° C., or from about 60° C. to about 90° C. or from about 70° C. to about 80° C.

The nonaqueous phase may comprise any form which is not aqueous, i.e., not prepared with water. By way of nonlimiting example, the nonaqueous phase may be a nonaqueous liquid, a solid or a powder.

The nonaqueous phase may comprise about 1.0% to 30.0% by weight of an emulsifier system. The emulsifier system comprises emulsifiers, fats and mixtures thereof. Generally, the emulsifier system comprises 0.5% to 20.0% (by weight of the final composition) of at least one emulsifier and 1.0% to 10.0% by weight (also of the final composition) of at least one fat. The function of the emulsifier is to prevent the oil or fat phase from separating from the polyols of the product. The emulsifier also provides good aeration in the buccal cavity during chewing. Emulsifiers work well to provide a smooth mouthfeel and help prevent the product from sticking to the packaging materials. However, there is a critical level of usage for the emulsifier beyond which the beneficial effect of the emulsifier will be negated. In other words, too high a percentage of emulsifiers will result in a reduction of the bite force of the stable solid delivery system and act to create a softer product.

Examples of emulsifiers that work well in the present inventive process include, without limitation, acetylated monoglycerides, glycerol esters, lecithin, de-oiled lecithin, enzyme-modified lecithins, purified lecithins, glycerol monostearate, polyglycerol esters, propylene glycol esters, sorbitan esters, polysorbate esters, sodium laurel sulfate, polyethylene glycols, sorbitol mono-, di- and tri-stearates, waxes and mixtures thereof. Selection of the proper emulsifier will depend on the desired characteristics of the final composition.

The emulsifier system also includes at least one fat component. The fats are chosen based on their solid fat index (SFI), active oxygen stability and melting characteristics at mammalian body temperature. The fat component of the emulsifier system acts to improve the pliability of the final composition. However, if the level of fats in the composition is too high, or the fats are added at too high of a temperature, separation or “oiling off” is observed during handling of the compositions. By adding the emulsifier system at the correct temperature, the oil-soluble compounds become embedded in the polyols, and form an oil-in-water emulsion.

Examples of fats that may be used in the emulsifier system include, without limitation, chocolate, cocoa butter, palm oil, canola oil, corn oil, sunflower oil, coconut oil, partially hydrogenated soybean oil, partially hydrogenated palm oil, partially hydrogenated coconut oil, partially hydrogenated canola oil, partially hydrogenated cottonseed oil, recinolate, and mixtures thereof. Selection of the fat component for use in the emulsifier system will depend on the desired characteristics of the final stable solid delivery system.

The compositions of the present invention may also contain one or more emulsifiers. Any substance known in the art as an emulsifier can be used, specifically, surfactants can be used. Categories of surfactants which can be used include, but are not limited to, hydrophilic surfactants, both ionic and nonionic, as well as lipophilic surfactants. Non-limiting examples of surfactants which can be used in the present invention include polyethoxylated fatty acids such as PEG 4-100 monolaurate, PEG-4 laurate, PEG-5 Stearate, and the like; PEG-Fatty Acid Diesters such as PEG-4 dilaurate, Peg 400 DL, PEG 1000 DS, and the like; PEG-Fatty Acid Mono- and Di-ester Mixtures such as PEG 4-150 mono, dilaurate, PEG 200-6000 mono dilaurate, PEG 4-150 mono distearate, and the like; Polyethylene Glycol Glycerol Fatty Acid Esters such as PEG-20 glyceryl laurate, PEG-40 glyceryl laurate, PEG-30 glyceryl oleate, and the like; Alcohol-Oil Transesterification Products such as PEG 20 castor oil, PEG-100 hydrogenated castor oil, PEG-60 corn glycerides, and the like; Polyglycerized fatty acids such as polyglyceryl-2 stearate, polyglyceryl-3 oleate, polyglyceryl-10 linoleate, and the like; Propylene Glycol Fatty Acid Esters such as propylene glycol monocaprylate, propylene glycol monooleate, propylene glycol dioctanoate, and the like; Mixtures of Propylene Glycol Esters-Glycerol Esters; Mono- and diglycerides such as monopalmitolein, 3-4 glyceryl monoleate, glyceryl monostearate, and the like; Sterol and Sterol Derivatives such as sitosterol, phytosterol, soya sterol and the like; Polyethylene Sorbitan Fatty Acid Esters such as sorbitan laurate; sorbitan monolaurate; sorbitan monostearate, and the like; Polyethylene Glycol Alkyl Ethers such as PEG-10 oleyl ether, PEG-10 cetyl ether, PEG-10 stearyl ether, and the like; Sugar Esters such as sucrose distearate/monostearate, sucrose monostearate, sucrose monopalmitate, and the like; Polyethylene Glycol Alkyl Phenols such as PEG-10-100 nonyl phenol, PEG-15-100 octyl phenol ether, and the like; Polyoxyethylene-Polyoxypropylene Block Copolymers such as Poloxamer 105 a=11 b=16, Poloxamer 185 a=19 b=30, Poloxamer 338 a=128 b=54, and the like; Sorbitan Fatty Acid Esters such as sorbitan monooleate, sorbitan sesquioleate, sorbitan monoisostearate, and the like; Lower Alcohol Fatty Acid Esters such as athyl oleate, isopropyl myristate, isopropyl linoleate, and the like; Ionic Surfactants such as sodium oleate, sodium taurocholate, palmitoyl carnitine, and the like; Unionized Ionizable Surfactants such as free fatty acids, bile acids, and the like; and Derivatives of Fat-Soluble Vitamins such as vitamins A, D, E, K, and the like.

The aqueous liquid phase is prepared by applying thermal energy, radiation, or mechanical force to an aqueous composition without substantial moisture loss such that the aqueous phase has reduced viscosity compared to the aqueous composition. For example, the aqueous phase may be prepared at a temperature ranging from about 80° F. to about 160° F. In some preferred aspects, the aqueous phase may be prepared at a temperature ranging from about 80° F. to about 240° F., or from about 80° F. to about 200° F., or from about 80° F. to about 180° F., from about 80° F. to about 160° F., or from about 80° F. to about 140° F.

When the aqueous phase is being prepared according to the above-described process, it is important to monitor two characteristics of the aqueous phase being formed: namely, moisture loss and viscosity reduction. The aqueous phase should be preferably prepared without substantial moisture loss. In one aspect, the substantial moisture loss is about 30% or less. In another aspect, the substantial moisture loss is about 25% or less. In a preferred aspect, the substantial moisture loss is about 20% or less, or about 15% or less, or about 10% or less, or about 5% or less, or about 1% or less.

The products prepared according to the present invention have reduced or substantially reduced moisture loss as described elsewhere. As a result, the products of the invention retain a high moisture content in such a way that the moisture is not easily lost even when subjected to high ambient temperatures as one may encounter during transportation and/or storage. For example even when the ambient temperatures rise to greater than 120° F., as in the case of a closed transportation container (i.e., in a closed truck or in a closed car trunk), the product was found to have not substantially lost its moisture as observed by its fairly good retention of its short structure, texture, and non-stickiness to the wrapper/container. The non-stickiness is quite surprising because several prior art products that do not retain moisture in the same way as the products of the present invention become gummy, lose their short structure, shape, and become sticky to the wrapper/container such that the product cannot be separated from the wrapper/container without losing some of the product by way of adhesion to the wrapper/container. This is an unexpected result.

To minimize moisture loss during preparation, the method may be performed under reduced pressure, or in a closed-system or both. These techniques are generally known in distillation, purification and pharmaceutical and confectionary arts.

The aqueous phase being formed will have a substantially reduced viscosity compared to the aqueous composition that is the starting material for making the aqueous phase. In one aspect, the substantially reduced viscosity represents a reduction in viscosity of from about 2 times to about 40 times. In other words, the aqueous phase will have a viscosity reduction of from about 2 times to about 40 times compared with the aqueous starting material. In another aspect, the substantially reduced viscosity represents a reduction in viscosity of from about 2 times to about 30 times. In some preferred aspects, the substantially reduced viscosity represents a reduction in viscosity of: from about 2 times to about 20 times; from about 2 times to about 10 times; from about 2 times to about 6 times. The degree of viscosity reduction depends on the aqueous material in question that is being subjected to energy input, such as heat. Thus, viscosity reduction for glycerin can be different in degree from that observed with corn syrup, for example.

The aqueous composition may be any material that can form an aqueous phase as it is or upon input of energy. For example, suitable materials may include: humectants, gums, syrups, polyols, or mixtures thereof. Examples of humectants that may be used include: glycerin, propylene glycol, or mixtures thereof. Examples of polyols may include: hydrogenated starch hydrolysate, isomalt, erythritol, polydextrose, maltitol, lactitol, glycerin, sorbitol, xylitol, mannitol, and syrups and mixtures thereof. Examples of gums that may be used include: Arabic, gelatin, starch, xanthan gum, gellan gum, or mixtures thereof. Examples of syrups may include: corn syrup, maple syrup, or mixtures thereof.

When polyols are used, generally, a mixture is preferred. It has been found that the polyols mixture may help to produce a base which is flexible and non-staling over time, and which does not stick to teeth or packaging materials. In some aspects, if stickiness is an issue, sorbitol content may be reduced to generally less than 2%. The polyols may be present in an amount from about 15% to 80% by weight.

The nonaqueous phase is contacted with the aqueous phase to obtain an emulsion. The emulsion formed by contacting the aqueous and nonaqueous phases may be a homogenous emulsion, i.e., an emulsion which is uniformly dispersed. The contacting may include activities such as mixing, applying, rolling, shaking, stirring, dispersing, etc such that the two phases come together and form an emulsion base. This emulsion base may be allowed to cool to room temperature or may be further processed to form a dosage form including but not limited to a tablet, granule, bead, confectionary unit of use, etc. Generally these forming methods are well known in the art. For example, the emulsion base may be compressed into a tablet, rope-pinched into a tablet, deposited into a specific predetermined geometric shape, or formulated into beads or granules.

The chewable emulsion base may be used to deliver nutriceuticals, food items (confectionary) or therapeutic agents or other active agents such as vitamins and minerals. The active agent may be uncoated, or coated. Coatings such as taste-masking, film coating, enteric coating, delayed-release coating, sustained-release coating, etc. The various coatings, coating materials and processes for achieving such coatings are well-known in the pharmaceutical and confectionary arts.

The active agent may be incorporated into the chewable base material at a temperature below that of either nonaqueous or aqueous phase formation. In some aspects, this low-temperature incorporation is desirable because the active may be thermo-labile (i.e., either degrades at higher temperatures or loses its functionality). Thus, the incorporation of the active agent into the chewable base material may occur a lower temperature than that of the preparation of the aqueous phase as well as at a lower temperature than the preparation of the nonaqueous phase.

To the emulsion base (and by extension, to the chewy product) additional ingredients may be added such as: fillers, binders, colorants, humectants, preservatives, taste-masking agents, sweeteners, and stabilizers or mixtures thereof.

In one aspect, the formed chewable composition may be coated with a taste-masking agent, an enteric coating material or a film-coating agent or combination thereof. Again, the various coatings, coating materials and processes for achieving such coatings are well-known in the pharmaceutical and confectionary arts.

The compositions of the present inventive subject matter may also include water in an amount from about 0% to 15% by weight of the final composition. In one aspect, the compositions also include a viscosity improvement agent. The viscosity improvement agents help improve the viscosity of the polyol/water mixture. An improved viscosity is important in providing a final composition that is chewy in nature. The viscosity improvement agent is present in amounts from about 0.1% to 10% by weight of the final composition. The viscosity improvement agent may include, without limitation, locust bean gum, guar gum, hydrolyzed guar gum (benefiber) carrageenan, starches, gum arabic, gelatin, agar, alginate, pectin and mixtures thereof.

In another preferred aspect, the compositions further comprise a bioadhesive agent. The bioadhesive agent helps the compositions be retained in the mouth during chewing, thus aiding in improving the oral hygiene of the user. Bioadhesive agents useful in the present inventive subject matter include, without limitation, hydroxypropylmethyl cellulose, ethyl cellulose, acrylic esters, polyvinyl acetates, alcohols and gums.

In general, the active ingredient is present in the inventive compositions in an amount from about 0.1% to about 70% by weight of the final composition. Preferably, the active ingredient is present in an amount from 1.0% to 50% by weight. One such preferred active ingredient is dietary fiber. The term “dietary fiber” is understood to mean the component of food which is non-digestible and non-metabolizable by humans. It is well known, however, that dietary fibers as they occur naturally in food sources also have associated with them a small digestible portion comprising fats, proteins, and carbohydrates.

Dietary fiber can be divided into two broad categories: insoluble dietary fiber and water soluble dietary fiber. For purposes of this invention, “insoluble dietary fiber” means the water insoluble portion of an edible material remaining after chemical and enzymatic treatment has removed proteins, fats and carbohydrates. For example, brans, celluloses, hemicellulases lignin and the like, are among those useful. “Soluble dietary fiber” means dietary fiber which is the water soluble portion of an edible material remaining after the chemical and enzymatic treatment has removed proteins, fats and carbohydrates. For example, pectin, guar gum, locust bean gum, gum arabic, karaya gum and others from the galacturonan and galactomannan classes; as well as psyllium seed gum, carageenan, konjac mannan, among others. These soluble fibers have been known to inhibit absorption of cholesterol in mammals, as well as re-absorption of bile salts. The mechanism for this benefit is believed to be three-fold in nature. First, the shear mass of the swelled fiber occludes the cholesterol and bile salts, thereby preventing absorption. Second, the fibers will absorb the cholesterol and bile salts, thereby physically transporting them out of the body. Finally, the fibers increase the transit time of stool bulk, which decreases the time in which absorption of cholesterol and bile salts can occur. Dietary fiber provides the bulking effect commonly associated with fiberous materials.

Useful dietary fiber substrates include noncellulosic polysaccharides, pectin, gums, algal polysaccharides, recently developed specialty maltodextrins, cellulose, hemicellulose, fructo-oligo saccharides, psyllium, lignin, mucilages and mixtures thereof. The dietary fiber is present in the compositions in amounts of about 0.1% to about 20% by weight. Further, the active agent may be selected from the group consisting of therapeutically active substances, stannol esters, vitamins, minerals, antacids, cough and cold medications, analgesics, cardiovasular medications, anti-smoking, psycho-therapeutics, antibiotics, and mixtures thereof.

Examples of therapeutically active substances that may be active agents in the present inventive process include, without limitation, antitussives, antihistamines, decongestants, alkaloids, mineral supplements, laxatives, vitamins, antacids, ion exchange resins, anti-cholesterolemics, antiarrhythmics, antipyretics, analgesics including acetaminophen, aspirin, non-asteroidal anti-inflammatory drugs (“NSAID”) and opioids, appetite suppressants, expectorants, anti-anxiety agents, anti-ulcer agents, anti-inflammatory substances, coronary dilators, cerebral dilators, peripheral vasodilators, anti-infectives, psycho-tropics, antimanics, stimulants, gastrointestinal agents, sedatives, anti-diarrheal preparations, anti-anginal drugs, vasodilators, anti-hypertensive drugs, vasoconstrictors, migraine treatments, antibiotics, tranquilizers, anti-psychotics, antitumor drugs, anticoagulants, antithrombotic drugs, hypnotics, anti-emetics, anti-nausants, anti-convulsants, neuromuscular drugs, hyper- and hypoglycemic spasmodics, uterine relaxants, mineral and nutritional additives, antiobesity drugs, anabolic drugs, erythropoetic drugs, antiasthmatics, cough suppressants, mucolytics, anti-uricemic drugs and mixtures thereof.

Further preferred nutritional active materials useful in the present inventive subject matter include, without limitation, calcium-containing materials such as calcium carbonate, stannol esters, hydroxycitric acid, vitamins, minerals, herbals, spices and mixtures thereof.

Examples of vitamins that are available as active ingredients include, without limitation, vitamin A (retinol), vitamin D (cholecalciferol), vitamin E group (alpha.-tocopherol and other tocopherols), vitamin K group (phylloquinones and menaquinones), thiamine (vitamin B₁), riboflavin (vitamin B₂), niacin, vitamin B₆ group, folic acid, vitamin B₁₂ (cobalamins), biotin, vitamin C (ascorbic acid), and mixtures thereof. The amount of vitamin or vitamins present in the final encapsulated product of the present inventive subject matter is dependent on the particular vitamin and is generally the United States' Department of Agriculture Recommended Daily Allowances (USRDA) for that vitamin. For example, if vitamin C is the active ingredient and the encapsulated product is being used in a confectionery or chewing gum targeting adults, the amount of vitamin C in the encapsulated product would be 60 milligrams, which is the USRDA of vitamin C for adults.

Examples of minerals that are available as active ingredients include, without limitation, calcium, magnesium, phosphorus, iron, zinc, iodine, selenium, potassium, copper, manganese, molybdenum and mixtures thereof. As is the case with vitamins, the amount of mineral or minerals present in the final encapsulated product of the present inventive subject matter is dependent on the particular mineral and is generally the USRDA for that mineral. For example, if iodine is the active ingredient and the encapsulated product is being used in a confectionery or chewing gum targeting adults, the amount of iodine in the encapsulated product would be 150 micrograms, which is the USRDA of iodine for adults.

Flavors may be chosen from natural and synthetic flavor liquids. Flavors useful in the present inventive process include, without limitation, chocolate, volatile oils, synthetic flavor oils, flavoring aromatics, oils, liquids, oleoresins or extracts derived from plants, leaves, flowers, fruits, stems and combinations thereof. A non-limiting list of examples include citrus oils such as lemon, orange, grape, lime and grapefruit and fruit essences including apple, pear, peach, grape, strawberry, raspberry, cherry, plum, pineapple, apricot or other fruit flavors.

Other useful flavorings include aldehydes and esters such as benzaldehyde (cherry, almond), citral, i.e., alphacitral (lemon, lime), neral, i.e., beta1-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), and 2-dodecenal (citrus, mandarin), and mixtures thereof.

In a preferred embodiment, a lemon-lime flavor for healthy chews comprises total liquid and powder flavors in an amount of 3.5%-4.0% by weight. Such flavors are refreshing, mouthwatering and cause salivation. Such flavors include caramel, vanilla and derivatives such as vanillan, lemon-lime liquid flavor, lemon-lime flavor encapsulation, freeze-dried orange crystals, and cream powder flavor.

In another preferred embodiment, a tropical flavor for healthy chews comprises a total tropical liquid and powdered flavor form in an amount of 3.6%-4.1% by weight. Such a flavor is refreshing, mouthwatering and causes salivation.

In yet another preferred embodiment, an oral hygiene composition for healthy chews comprises total liquid and powder flavors in an amount of 2.9%-3.5% by weight. Such flavors include cool-mint flavor, menthol flavor encapsulation, eucalyptus oil, peppermint liquid, and peppermint flavor encapsulation.

Further examples of flavors useful in the inventive process include, without limitation, beef flavorings, chicken flavorings, rice flavorings, lamb flavorings, pork flavorings, seafood flavorings, and mixtures thereof.

The sweeteners may be chosen from the following non-limiting list: saccharin and its various salts such as the sodium salt; dipeptide sweeteners such as aspartame; dihydrochalcone compounds, glycyrrhizin; Stevia rebaudiana (Stevioside); chloro derivatives of sucrose such as sucralose; sugar alcohols such as sorbitol, mannitol, zylitol, and the like. Also contemplated are hydrogenated starch hydrolysates and synthetic sweetener 3,6-dihydro-6-methyl-1-1-1,2,3-oxathiazin-4-one-2,2-dioxide, particularly the potassium salt (acesulfame-K) and sodium and calcium salts thereof. Other sweeteners may also be used.

Thus, in one aspect, the composition prepared according to the present methods comprise a mixture of at least two polyols present in an amount from about 15% to 80% by weight, an emulsifier system present in an amount from about 1.0% to 30% by weight, an active ingredient in an amount from about 0.1% to 70% by weight, a bioadhesive agent for improving oral hygiene and water in an amount from 0.0% to 15% by weight. Each component is as defined above and given the same meaning as above.

In one aspect, the first step in the method of the present inventive subject matter comprises mixing from about 15% to 80% by weight of at least two polyols, as defined above, with water present in an amount of about 0.0% to 15% by weight. The polyols are mixed in a conventional mixer or cooker. The mixer or cooker may be a batch or continuous process mixer or cooker. The mixture is then heated to a temperature of about 200-260° F. This temperature is maintained for about 5 to 10 minutes, allowing some of the moisture to be evaporated off. When the moisture level is about 12% by weight water, or a Brix of about 87 is attained, the mixture is cooled to a temperature of about 175-240° F.

To the cooled mixture is added about 1.0% to 30% by weight of an emulsifier system. Again, the emulsifier system is identical to the emulsifier system define above with respect to the compositions of the present inventive subject matter. The addition of the emulsifier system to the cooled mixture forms a further mixture. The further mixture is then cooled, and the above active agents, in amounts of up to 0.1% to 70% by weight are added, thus forming the composition. Other ingredients, such as flavors, colors, sweeteners, bulking agents, and the like may also be added at this time. The final mixture is then formed into the final product and allowed to set up.

In a preferred embodiment of this invention, a viscosity improvement agent is added to the polyol/water mixture in the first step of the process. The viscosity improvement agent is added in amount of about 0.1% to 10% by weight. The viscosity improvement agent is as defined above.

In still another preferred embodiment of this invention, a bioadhesive agent is added with the active agent in the last step of the inventive method. The bioadhesive agent is as defined above.

Generally, the emulsifiers, fats and/or waxes of the present invention may be melted at a temperature of about 120° F. to about 200° F., more preferably about 140° F. to about 175° F. For example, some emulsifiers may be best melted at about 140° F. to about 150° F., while others may be best melted at about 165° F. to about 175° F. The melted emulsifiers, fats and/or waxes will then be mixed with other components as indicated herein. The determination of proper melting point for the emulsifiers, fats and/or waxes is well within the purvue of one of skill in the art.

In a yet further embodiment of the present inventive subject matter, the inventive compounds may be made by the following an alternative method. The emulsifiers in the composition may be melted independent of the polyols or proteins. Generally, the emulsifiers will be melted at a temperature of about 110° F. The melted emulsifiers will then be mixed with other components including the active agents, colors, sweeteners, flavors, etc., at a relatively constant temperature until homogeneous. Upon the mixture becoming homogeneous, the polyols and/or proteins are then added to the mixture to form a further mixture. The further mixture is mixed until homogeneous, after which the product is formed and allowed to set up.

In yet a still further embodiment of the method of the present inventive subject matter, the heating of the mixture in the second step may be done under vacuum, thus allowing for a shorter time needed for mixing the polyols with the water.

Each of the methods of manufacturing the chewy compositions of the present invention allow for the retention of moisture in the composition. The retention of the moisture allows for other ingredients, such as flavors, active agents, etc., to be added at a later time. The incorporation of the active agents after the composition has been formed provides great flexibility for the uses of the present inventive process. For example, the active agents may be incorporated into the compositions immediately after cooling the composition to a temperature around 110° F., thus resulting in a final product which may be formed and packaged in conventional manners for shipment and delivery to a customer. In this example, the active agents and composition are mixed in the same plant or facility in which the composition was prepared. Also, the preparation of the composition and the mixing of the active agent are done within a short period of time (within a couple of hours) of each other.

Another advantage of the composition is that the active agents may be incorporated therein at a later time. For example, the composition may be prepared by the inventive process and intermediately packaged for storage. Then at a later date (days, weeks or months after initial preparation of the delivery system), the composition is reheated and the active agents are incorporated into the composition, resulting in a final product that is formed and packaged by conventional means and delivered to the customer. The amount of time allowed between the initial manufacture of the chewy composition and incorporation of additional ingredients, e.g., active ingredients or flavorants, can vary from less than one day to about one year. In one embodiment the chewy composition is stored for a period of 1 day prior to the incorporation of the additional ingredients. In another embodiment the chewy composition is stored for a period of 1 week prior to the incorporation of the additional ingredients. In yet another embodiment the chewy composition is stored for a period of about one month prior to the incorporation of the additional ingredients. In a further embodiment, the chewy composition is stored for a period of about three months prior to the incorporation of the additional ingredients. In still another embodiment, the chewy composition is stored for a period of about 6 months prior to the incorporation of additional ingredients. In another embodiment, the chewy composition is stored for a period of about 1 year prior to incorporation of additional ingredients.

It is contemplated within the scope of the present inventive process that this later incorporation of the active agents into the composition may take place at the same site as did the preparation of the composition, or it may take place at a remote location. The remote location may be another building within the same complex as the facility in which the delivery system was prepared, or it may be at a location that is away from the delivery system production site. Of course, if the remote location is a site that is away from the production site, then the intermediately packaged composition must be transported to the remote location. The present inventive subject matter contemplates all modes of transporting the composition to the remote location, including without limitation, by truck or other automotive vehicle, airplane, train, or ship.

In a preferred embodiment of the present inventive process, the composition is formed into a desired shape and cooled to room temperature. After a period of time, the composition is reheated to a temperature of about 110° F. and at least one active agent is mixed therewith to form the final product. The reheating of the composition may take place at a remote location, as is discussed above.

In another preferred embodiment of the present inventive process, the composition is formed into a desired shape and cooled to room temperature, after which the composition is packaged for transport to a remote location. After the composition is transported to the remote location, it is removed from the packaging and heated to a temperature of about 110° F. and mixed with at least one therapeutically active substance to form a pharmaceutical composition.

Processes for preparing solid delivery systems are described, for example, in PCT/US02/19334 (published as WO 03/000229), which is a continuation application of U.S. application Ser. No. 09/886,473, (published as U.S. Publication No. 2002/0197323), both of which are herein incorporated by reference in their entirety.

In another embodiment of the present invention, compositions prepared by the methods set forth supra are contemplated. The compositions of the present invention may comprise:

an active ingredient; liquids; and an emulsifier system, wherein the emulsifier system comprises an emulsifier, a fat, and/or mixtures thereof. Preferably, the compositions of the present invention may further comprise a base, may further comprise a bulk material, and/or may further comprise cellulose compounds. The compositions may also include water. The compositions may also include a viscosity improving agent as needed. The compositions may also include a bioadhesive agent as needed.

The compositions of the present invention may further comprise flavors, sweeteners, and or colors.

By way of non-limiting example, a composition of the present invention for the administration of fiber may comprise fiber as the active ingredient, maltitol syrup and glycerin as liquids, soy lecithin and distilled mono- and di-glycerides as emulsifiers, crystalline maltitol and non-fat dry milk, chocolate liqueor as flavoring, and Acesulfame-K as sweetener.

Bilayer chewable compositions are also contemplated by the present invention. Such compositions contain two layers, prepared separately, as illustrated by Example 16 herein. Each layer may contain a different active ingredient, thus providing a vehicle for delivery more than one active ingredient in a single dosage form, without the two actives having to be in contact with one another.

The following examples are given to illustrate the invention, but are not deemed to be limiting thereof. All percentages given throughout the specification are based upon weight unless otherwise indicated.

### EXAMPLES

### Example 1

The composition of this Example is given in Table 1. The polyols were mixed with water in a conventional mixer or cooker. The mixer or cooker may be a batch or continuous process mixer or cooker. The mixture was then heated to a temperature of about 200° F.-260° F. This temperature was maintained for about 5 to 10 minutes, allowing some of the moisture to be evaporated off. When the moisture level was about 12% by weight water, or a Brix of about 87 was attained, the mixture is cooled to a temperature of about 175° F.-240° F. The moisture loss and viscosity reduction were measured.

To the cooled mixture was added the emulsifier system to form a further mixture. The further mixture was then cooled, and the active agents were added, thus forming the composition. Other ingredients, such as flavors, colors, sweeteners, bulking agents, and the like may also be added at this time. The final mixture was then formed into the final product and allowed to set up. The moisture loss and viscosity reduction were measured.

TABLE 1
Fiber Chew 1.5 g Chocolate Flavor Composition
Piece weight: - 5.0 grams

| S. No. | Ingredients | mg/Dose |
|---|---|---|
| | Actives | |
| 1. | Soluble fiber (Maltodextrin, fibersol, | 1500.00 |
| | Inulin, Carboxy Methyl Cellulose, Sodium | |
| | CMC) or Insoluble fiber (Microcrystalline | |
| | Cellulose) or Blend | |
| | Liquids | |
| 2. | Maltitol Syrup | 1050.00 |
| 3. | Glycerin 99.6% | 250.00 |
| | Emulsifiers | |
| 4. | Soy Lecithin | 37.50 |
| 5. | Distilled Mono Glycerides (Melting Point: | 150.00 |
| | 150-160° F.) | |
| | Bulk Material | |
| 6. | Crystalline Maltitol | 1007.50 |
| | Dry Milk Solids | |
| 7. | Non Fat Dry Milk, LH grade | 300.00 |
| | Flavors | |
| 8. | Chocolate Liquor | 500.00 |
| 9. | Other flavors | 200.00 |
| | Intense Sweeteners | |
| 10. | Acesulfame-K | 5.00 |
| | Total | 5000 |

### Example 2

The general procedure of Example 1 was followed using the composition of Table 2 to prepare nattokinase soft-chew tablets. The moisture loss and viscosity reduction were measured.

TABLE 2
Nattokinase chew Composition
Piece weight: - 5.0 grams

| S. No. | Ingredients | mg/Dose |
|---|---|---|
| | Actives | |
| 1. | Nattokinase | 1500 |
| 2. | Vitamin Blend contains B3, B6, Folic acid | 345 |
| | and B12 | |
| | Liquids/Binders | |
| 3. | Maltitol Syrup | 1025 |
| 4. | Glycerin 99.6% | 200 |
| | Emulsifiers | |
| 5. | Soy Lecithin | 37.5 |
| 6. | Distilled Mono Glycerides (Melting Point: | 105 |
| | 150-160° F.) | |
| | Bulk Material | |
| 7. | Crystalline Maltitol | 82.5 |
| 8. | Soluble fiber (Maltodextrin, fibersol, | 750 |
| | Inulin, Carboxy Methyl Cellulose, Sodium | |
| | CMC) or Insoluble fiber (Microcrystalline | |
| | Cellulose) or Blend | |
| | Dry Milk Solids | |
| 9. | Non Fat Dry Milk, LH grade | 300 |
| | Flavors | |
| 10. | Chocolate Liquor | 450 |
| 11. | Other flavors | 200 |
| | Intense Sweeteners | |
| 12. | Acesulfame-K | 5 |
| | Total | 5000 |

### Example 3

The general procedure of Example 1 was followed using the composition of Table 3 to prepare soft-chew tablets comprising two active agents: calcium and insulin. The moisture loss and viscosity reduction were measured.

TABLE 3
Calcium + fiber Chew Composition
Piece weight: - 5.0 grams

| S. No. | Ingredients | mg/Dose |
|---|---|---|
| | Actives | |
| 1. | Calcium Carbonate or different salts of | 1116.5 |
| | calcium | |
| 2. | Soluble fiber (Maltodextrin, fibersol, | 869.6 |
| | Inulin, Carboxy Methyl Cellulose, Sodium | |
| | CMC) or Insoluble fiber (Microcrystalline | |
| | Cellulose) or Blend | |
| | Liquids/Binders | |
| 3. | Maltitol Syrup | 1100 |
| 4. | Glycerin 99.6% | 175 |
| | Emulsifiers | |
| 5. | Soy Lecithin | 37.5 |
| 6. | Distilled Mono Glycerides (Melting Point: | 105 |
| | 150-160° F.) | |
| 7. | Partially hydrogenated Soyabean and | 73.5 |
| | Cotton seed oil (Melting Point: 160-175 F.) | |
| | Bulk Material | |
| 8. | Crystalline Maltitol | 560.1 |
| | Dry Milk Solids | |
| 9. | Non Fat Dry Milk, LH grade | 250 |
| | Flavors | |
| 10. | Chocolate Liquor | 500 |
| 11. | Other flavors | 200 |
| | Intense Sweeteners | |
| 12. | Acesulfame-K | 5 |
| 13. | Aspartame | 7.5 |
| | Total | 5000 |

### Example 4

The general procedure of Example 1 was followed using the composition of Table 4 to prepare calcium carbonate soft-chew tablets. The moisture loss and viscosity reduction were measured.

TABLE 4
Calcium Chew Composition
Piece weight: - 5.0 grams

| S. No. | Ingredients | mg/Dose |
|---|---|---|
| | Actives | |
| 1. | Calcium Carbonate or different salts of | 1177.00 |
| | calcium | |
| | Liquids | |
| 2. | Maltitol Syrup | 950.00 |
| 3. | Glycerin 99.6% | 175.00 |
| | Emulsifiers | |
| 4. | Soy Lecithin | 88.50 |
| 5. | Distilled Mono Glycerides(Melting Point: | 255.00 |
| | 150-160° F.) | |
| | Diluents | |
| 6. | Maltodextrin/Corn Syrup Solids | 694.50 |
| 7. | Sugar | 929.00 |
| | Dry Milk Solids | |
| 8. | Non Fat Dry Milk, LH grade | 600.00 |
| | Cellulose Compounds | |
| 9. | Hydroxypropyl Methyl Cellulose | 25.00 |
| | Flavors | |
| 10. | Flavors | 100.00 |
| | Sweeteners | |
| 11. | Acesulfame-K | 5.00 |
| | Colors | |
| 12. | Red # lake (Optional) | 1.00 |
| | Total | 5000 |

### Example 5

The general procedure of Example 1 was followed using the composition of Table 5 to prepare elemental zinc soft-chew tablets. The moisture loss and viscosity reduction were measured.

TABLE 5
Zinc Vapor Action Chew Composition
Piece weight: - 5.0 grams

| S. No. | Ingredients | mg/Dose |
|---|---|---|
| | Actives | |
| 1. | Zinc Acetate Dihydrate or different salts | 10.5 |
| | of Zinc | |
| | Liquids | |
| 2. | Maltitol Syrup | 925 |
| 3. | Glycerin 99.6% | 175 |
| | Emulsifiers | |
| 4. | Soy Lecithin | 37.5 |
| 5. | Distilled Mono Glycerides (Melting Point: | 250 |
| | 150-160° F.) | |
| | Base | |
| 6. | Maltodextrin/Corn Syrup Solids | 1000 |
| | Cellulose Compounds | |
| 7. | Hydroxypropyl Methyl Cellulose | 25 |
| | microcrystalline cellulose | 625 |
| | Flavors | |
| 8. | Menthol Encapsulation | 27.5 |
| 9. | cool Flavor | 5.5 |
| 10. | Flavors | 100 |
| | Sweeteners | |
| 11. | Sugar | 1818 |
| | Colors | |
| 12. | Green Color Blend (Optional) | 1 |
| | Total | 5000 |

### Example 6

The general procedure of Example 1 was followed using the composition of Table 6 to prepare two actives (zinc acetate and zinc gluconate) containing soft-chew tablets with strawberry flavoring. The moisture loss and viscosity reduction were measured.

TABLE 6
Zinc Acetate + Zinc Gluconate chewComposition
Piece weight: - 5.0 grams

| S. No. | Ingredients | mg/Dose |
|---|---|---|
| | Actives | |
| 1. | Zinc Acetate dihydrate | 27.5 |
| 2. | Zinc Gluconate or different salts of Zinc | 19 |
| | Liquids | |
| 3. | Maltitol Syrup | 950 |
| 4. | Glycerin 99.6% | 100 |
| | Emulsifiers | |
| 5. | Soy Lecithin | 38.5 |
| 6. | Distilled Mono Glycerides (Melting Point: | 250 |
| | 150-160° F.) | |
| 7. | Partially hydrogenated Soyabean and | 125 |
| | Cotton seed oil(Melting Point: 160-175 F.) | |
| | Base | |
| 8. | Maltodextrin/Corn Syrup Solids | 1000 |
| | Cellulose Compounds | |
| 9. | Hydroxypropyl Methyl Cellulose | 25 |
| | Methyl Cellulose | 625 |
| | Flavors | |
| 10. | Strawberry flavor | 125 |
| | Sweeteners | |
| 11. | Sugar | 1713 |
| | Colors | |
| 12. | Red#40 dye (Optional) | 2 |
| | Total | 5000 |

### Example 7

The general procedure of Example 1 was followed using the composition of Table 7 to prepare soft-chew tablets containing three actives: elemental zinc, copper chlorophyllin and copper gluconate. The moisture loss and viscosity reduction were measured.

TABLE 7
Oral care chew composition
Piece weight: - 5.0 grams

| S. No. | Ingredients | mg/Dose |
|---|---|---|
| | Actives | |
| 1. | Zinc Acetate dihydrate or different salts of | 27.50 |
| | zinc | |
| 2. | Sodium Copper Chlorophyllin USP | 12.50 |
| 3. | Copper Gluconate USP | 10.00 |
| | Liquids | |
| 4. | Maltitol Syrup | 975.00 |
| 5. | Glycerin 99.6% | 175.00 |
| | Emulsifiers | |
| 6. | Soy Lecithin | 50.00 |
| 7. | Distilled Mono Glycerides (Melting Point: | 250.00 |
| | 150-160° F.) | |
| 8. | Polysorbate 80 | 0.50 |
| 9. | Sodium Lauryl Sulfate | 1.00 |
| 10. | Sodium Stearate | 0.50 |
| 11. | Sorbitan Monostearate | 0.50 |
| | Base | |
| 12. | Maltodextrin/Corn Syrup Solids | 1005.00 |
| | Cellulose Compounds | |
| 13. | Hydroxypropyl Methyl Cellulose | 25.00 |
| | Microcrystalline cellulose | 625.00 |
| | Flavors | |
| 14 | Flavor Actives | 100 |
| 15 | Other flavors | 100 |
| | Sweeteners | |
| 16. | Sugar | 1370.5 |
| | Colors | |
| 17. | Green Color Blend (Optional) | 2.00 |
| | Total | 5000 |

### Example 8

The general procedure of Example 1 was followed using the composition of Table 8 to prepare soft-chew tablets containing Fibersol as the active ingredient. The moisture loss and viscosity reduction were measured.

TABLE 8
Product: Fibersol 1.5 g - Chocolate flavor
Piece weight: 5.50 gm

| S. NO | Material Name | mg/unit |
|---|---|---|
| | Active Ingredients | |
| 1 | Fibersol | 1666.50 |
| | Emulsifiers/Fats/Waxes | |
| 2 | Mono and Di Glyceride (Melting Point: 150-160° F.) | 275.00 |
| | Diluents | |
| 3 | Chocolate Liquor | 495.00 |
| 4 | Crystalline Maltitol | 1174.25 |
| | Liquid System | |
| 5 | Glycerin | 165.0 |
| 6 | Maltitol Syrup | 1182.50 |
| | Flavor system | |
| 7 | Non Fat Dry Milk | 330.00 |
| 8 | Caramel Flavor | 22.00 |
| 9 | Vanilla Flavor | 11.00 |
| 10 | Chocolate Powder | 19.25 |
| 11 | Chocolate fudge flavor liquid | 27.50 |
| 12 | milk flavor | 16.50 |
| 13 | Cream flavor powder | 27.50 |
| 14 | Cocoa Powder | 82.50 |
| | Intense Sweetener | |
| 15 | Sucralose | 5.50 |
| | Color | |
| | TOTAL | 5500.00 |

### Example 9

The general procedure of Example 1 was followed using the composition of Table 9 to prepare soft-chew tablets containing calcium carbonate as the active ingredient. The moisture loss and viscosity reduction were measured.

TABLE 9
Product Name: Extra Strength Antacid Soft Chew (Calcium
Carbonate 1177 mg) - Wild Cherry Flavor
Piece weight: 5.0 g.

| S. NO | Material Name | mg/unit |
|---|---|---|
| | Active Ingredients | |
| 1 | Calcium Carbonate | 1177.00 |
| | Emulsifiers/Fats/Waxes | |
| 2 | Mono and Di Glyceride (Melting Point: 150-160° F.) | 350.00 |
| | Diluents | |
| 3 | Confectioners Sugar | 664.00 |
| 4 | Maltodextrin | 930.00 |
| | Liquid Binding System | |
| 5 | Hypromellose | 25.00 |
| | Liquid System | |
| 6 | Glycerin | 175.00 |
| 7 | Maltitol Syrup | 1050.00 |
| | Flavor system | |
| 8 | Wild Cherry Flavor | 50.00 |
| 9 | Cream Flavor | 25.00 |
| 10 | Non Fat Dry Milk | 500.00 |
| 11 | Cherry Flavor | 12.50 |
| 12 | Vanilla Flav. | 15.00 |
| 13 | Natural Milk Flavor | 20.00 |
| | Intense Sweetener | |
| 14 | Sucralose | 5.00 |
| | Color | |
| 15 | Red #40 | 1.50 |
| | TOTAL | 5000.00 |

### Example 10

The general procedure of Example 1 was followed using the composition of Table 10 to prepare soft-chew tablets containing two active ingredients, benzocaine and menthol. The moisture loss and viscosity reduction were measured.

TABLE 10
Product Name: Benzocaine 6 mg and Menthol 10 mg Soft Chew
Piece weight: 3.0 g.

| S. NO | Material Name | mg/unit |
|---|---|---|
| | Active Ingredients | |
| 1 | Benzocaine | 6.00 |
| 2 | Spray Dried Menthol | 50.10 |
| | Emulsifiers/Fats/Waxes | |
| 3 | Mono & DiGlycerides (Melting Point: 140-150° F.) | 210.00 |
| 4 | Distilled Mono Glyceride (Melting Point: 150-160° F.) | 60.00 |
| | Diluents | |
| 5 | Confectioners Sugar | 1347.90 |
| 6 | Maltodextrin | 600.00 |
| | Liquid Binding System | |
| 7 | Hypromellose | 15.00 |
| | Liquid System | |
| 8 | Glycerin | 90.00 |
| 9 | Maltitol Syrup | 600.00 |
| | Flavor system | |
| 10 | Eucalyptus Oil | 3.30 |
| 11 | Peppermint Oil | 12.30 |
| 12 | Cool flavor | 4.20 |
| | Color | |
| 13 | Green color | 1.20 |
| | TOTAL | 3000.00 |

### Example 11

The general procedure of Example 1 was followed using the composition of Table 11 to prepare soft-chew tablets containing benzocaine and menthol as the active ingredient. The moisture loss and viscosity reduction were measured.

TABLE 11
Product Name: Benzocaine 6 mg and Menthol 10 mg Soft Chew
Piece weight: 5.0 g.

| S. NO | Material Name | mg/unit |
|---|---|---|
| | Active Ingredients | |
| 1 | Benzocaine | 6.00 |
| 2 | Spray Dried Menthol | 50.00 |
| | Emulsifiers/Fats/Waxes | |
| 3 | Mono & DiGlycerides (Melting Point: 140-150° F.) | 350.00 |
| 4 | Distilled Mono Glyceride (Melting Point: 150-160° F.) | 100.00 |
| | Diluents | |
| 5 | Confectioners Sugar | 2284.00 |
| 6 | Maltodextrin | 1000.00 |
| | Liquid Binding System | |
| 7 | Hypromellose | 25.00 |
| | Liquid System | |
| 8 | Glycerin | 150.00 |
| 9 | Maltitol Syrup | 1000.00 |
| | Flavor system | |
| 10 | Eucalyptus Oil | 5.50 |
| 11 | Peppermint Oil | 20.50 |
| 12 | Cool flavor | 7.00 |
| | Color | |
| 13 | Green color | 2.00 |
| | TOTAL | 5000.00 |

### Example 12

The general procedure of Example 1 was followed using the composition of Table 12 to prepare soft-chew tablets containing two active ingredients, glucosamine and chondroitin sulfate. The moisture loss and viscosity reduction were measured.

TABLE 12
Product: Soft Glucosamine HCl (750 mg) & Chondroitin
sulphate (600 mg)chew - Lemon lime flavor
Piece weight: 5.50 gm

| S. NO | Material Name | mg/unit |
|---|---|---|
| | Active Ingredients | |
| 1 | Encapsulated Glucosamine HCl 65.0% | 1155.00 |
| 2 | Chondroitin Sulfate Sodium | 599.50 |
| | Emulsifiers/Fats/Waxes | |
| 3 | Mono and Di Glyceride (Melting Point: 140-150° F.) | 467.50 |
| | Diluents | |
| 4 | Crystalline Maltitol | 1530.65 |
| | Liquid System | |
| 5 | Glycerin | 192.50 |
| 6 | Maltitol Syrup | 1210.00 |
| | Flavor system | |
| 7 | Vanilla Flav. | 22.00 |
| 8 | Cream Flavored Powder | 55.00 |
| 9 | lemon lime spray Dried Flavor | 82.50 |
| 10 | Orange crystals | 55.00 |
| | Intense Sweetener | |
| 11 | Sucralose | 5.50 |
| | Salivating Agent | |
| 12 | Citric Acid fine granular | 123.75 |
| | Color | |
| 13 | Yellow #5 | 1.10 |
| | | 5500 |

### Example 13

The general procedure of Example 1 was followed using the composition of Table 13 to prepare soft-chew tablets containing zinc as the active ingredient. The moisture loss and viscosity reduction were measured.

Product Name: Zinc Vapor Action Soft Chew (11 mg of Elemental Zinc)
Piece weight: 3.0 g.

| S. NO | Material Name | mg/unit |
|---|---|---|
| | Active Ingredients | |
| 1 | Milled Zinc Acetate Dihydrate | 27.60 |
| 2 | Zinc Gluconate Dihydrate | 21.60 |
| | Emulsifiers/Fats/Waxes | |
| 3 | Mono and Di Glyceride (Melting Point: 140-150° F.) | 210.00 |
| 4 | Hydrogenated Soy Bean Oil (Melting Point: 160-175 | 15.00 |
| | F.) | |
| 5 | Distilled Mono Glyceride (Melting Point: 150-160° F.) | 60.00 |
| 6 | Lecithin | 3.00 |
| | Diluents | |
| 7 | Confectioners Sugar | 1312.80 |
| 8 | Maltodextrin | 600.00 |
| | Liquid Binding System | |
| 9 | Hypromellose | 15.00 |
| | Liquid System | |
| 10 | Glycerin | 90.00 |
| 11 | Maltitol Syrup | 600.00 |
| | Flavor system | |
| 12 | Spray Dried Menthol | 24.00 |
| 13 | Eucalyptus Oil | 3.30 |
| 14 | Peppermint Oil | 12.30 |
| 15 | Cool flavor | 4.20 |
| | Color | |
| 16 | Green color | 1.20 |
| | TOTAL | 3000.00 |

### Example 14

The general procedure of Example 1 was followed using the composition of Table 14 to prepare soft-chew tablets containing Fibersol as the active ingredient. The moisture loss and viscosity reduction were measured.

TABLE 14
Product Name: Zinc Vapor Action Soft Chew (11 mg of Elemental Zinc)
Piece weight: 5.0 g.

| S. NO | Material Name | mg/unit |
|---|---|---|
| | Active Ingredients | |
| 1 | Milled Zinc Acetate Dihydrate | 27.50 |
| 2 | Zinc Gluconate Dihydrate | 21.50 |
| | Emulsifiers/Fats/Waxes | |
| 3 | Mono and Di Glyceride (Melting Point: 140-150° F.) | 350.00 |
| 4 | Hydrogenated Soy Bean Oil (Melting Point: 160-175 | 25.00 |
| | F.) | |
| 5 | Distilled Mono Glyceride (Melting Point: 150-160° F.) | 100.00 |
| 6 | Lecithin | 5.00 |
| | Diluents | |
| 7 | Confectioners Sugar | 2221.00 |
| 8 | Maltodextrin | 1000.00 |
| | Liquid Binding System | |
| 9 | Hypromellose | 25.00 |
| | Liquid System | |
| 10 | Glycerin | 150.00 |
| 11 | Maltitol Syrup | 1000.00 |
| | Flavor system | |
| 12 | Spray Dried Menthol | 40.00 |
| 13 | Eucalyptus Oil | 5.50 |
| 14 | Peppermint Oil | 20.50 |
| 15 | Cool flavor | 7.00 |
| | Color | |
| 16 | Green color | 2.00 |
| | TOTAL | 5000.00 |

### Example 15

The general procedure of Example 1 was followed using the composition of Table 15 to prepare soft-chew tablets containing two active ingredients, caffeine and taurine. The moisture loss and viscosity reduction were measured.

TABLE 15
Product: Soft Caffeine 40 mg & Taurine 10 mg chew - Orange flavor
Piece weight: 5.50 gm

| S. NO | Material Name | mg/unit |
|---|---|---|
| | Active Ingredients | |
| 1 | Encapsulated Caffeine 40% | 100.10 |
| 2 | L- Taurine 98.5% | 9.90 |
| | Emulsifiers/Fats/Waxes | |
| 3 | Mono and Di Glyceride (Melting Point: 140-150° F.) | 467.50 |
| | Diluents | |
| 4 | Crystalline Maltitol | 3175.15 |
| | Liquid System | |
| 5 | Glycerin | 192.50 |
| 6 | Maltitol Syrup | 1210.00 |
| | Flavor system | |
| 7 | Vanilla Flav. | 22.00 |
| 8 | Cream Flavored Powder | 55.00 |
| 9 | Orange Flavor | 82.50 |
| 10 | Orange crystals | 55.00 |
| | Intense Sweetener | |
| 11 | Sucralose | 5.50 |
| | Salivating Agent | |
| 12 | Citric Acid fine granular | 123.75 |
| | Color | |
| 13 | Yellow #6 | 1.10 |
| | | 5500 |

### Example 16

The general procedure of Example 1 was followed using the composition of Table 16 (first layer) and 17 (second layer) to prepare bilayer soft-chew tablets containing zinc as the active ingredient. The moisture loss and viscosity reduction were measured.

TABLE 16
Product Name: Zinc 10.5 mg & Vitamin C 60 mg
Chew - Cream and Orange flavor Swirl
Piece weight: 5.0 g. Layer 1

| S. NO | Material Name | mg/unit |
|---|---|---|
| | Active Ingredients | |
| 1 | Milled Zinc Acetate Dihydrate | 27.00 |
| 2 | Zinc Gluconate Dihydrate | 18.50 |
| | Emulsifiers/Fats/Waxes | |
| 3 | Mono and Di Glyceride (Melting Point: 140-150° F.) | 175.00 |
| | Diluents | |
| 4 | Confectioners Sugar | 903.75 |
| 5 | Maltodextrin | 375.00 |
| | Liquid System | |
| 6 | Glycerin | 75.00 |
| 7 | Maltitol Syrup | 550.00 |
| | Flavor system | |
| 8 | Orange Flavor | 62.50 |
| 9 | Orange Crystals | 25.00 |
| 10 | Cream Flavor | 25.00 |
| 11 | Non Fat Dry Milk | 250.00 |
| 12 | Vanilla Flavor | 10.00 |
| | Intense Sweetener | |
| 13 | Sucralose | 2.50 |
| | Color | |
| 14 | Yellow # 5 | 0.75 |
| | TOTAL | 2500.00 |

TABLE 17
Product Name: Zinc 10.5 mg & Vitamin C 60 mg
Chew - Cream and Orange flavor Swirl
Piece weight: 5.0 g. Layer 2

| S. NO | Material Name | mg/unit |
|---|---|---|
| | Active Ingredients | |
| 1 | Encapsulated Vitamin C 64.22% | 117.00 |
| | Emulsifiers/Fats/Waxes | |
| 2 | Mono and Di Glyceride (Melting Point: 140-150° F.) | 175.00 |
| | Diluents | |
| 3 | Confectioners Sugar | 832.25 |
| 4 | Maltodextrin | 375.00 |
| | Liquid System | |
| 5 | Glycerin | 75.00 |
| 6 | Maltitol Syrup | 550.00 |
| | Flavor system | |
| 7 | Orange Flavor | 62.50 |
| 8 | Orange Crystals | 25.00 |
| 9 | Cream Flavor | 25.00 |
| 10 | Non Fat Dry Milk | 250.00 |
| 11 | Vanilla Flavor | 10.00 |
| | Intense Sweetener | |
| 12 | Sucralose | 2.50 |
| | Color | |
| 13 | Red# 40 | 0.75 |
| | TOTAL | 2500.00 |

### Example 17

The general procedure of Example 1 was followed using the composition of Table 18 to prepare soft-chew tablets containing zinc as the active ingredient. The moisture loss and viscosity reduction were measured.

TABLE 18
Product Name: Zinc Chew- Strawberry Flavor
Piece weight: 5.0 g.

| S. NO | Material Name | QTY % |
|---|---|---|
| | Active Ingredients | |
| 1 | Milled Zinc Acetate Dihydrate | 0.55 |
| 2 | Zinc Gluconate Dihydrate | 0.38 |
| | Emulsifiers/Fats/Waxes | |
| 3 | Mono and Di Glyceride (Melting Point: 140-150° F.) | 7.00 |
| 4 | Hydrogenated Soy Bean Oil (Melting Point: 160-175 | 0.50 |
| | F.) | |
| 5 | Distilled Mono Glyceride (Melting Point: 150-160° F.) | 2.00 |
| 6 | Lecithin | 0.10 |
| | Diluents | |
| 7 | Confectioners Sugar | 45.43 |
| 8 | Maltodextrin | 20.00 |
| | Liquid Binding System | |
| 9 | Hypromellose | 0.50 |
| | Liquid System | |
| 10 | Glycerin | 2.50 |
| 11 | Maltitol Syrup | 18.50 |
| | Flavor system | |
| 12 | Strawberry Flavor | 2.50 |
| | Color | |
| 13 | Red # 40 | .04 |
| | TOTAL | 100.00 |

### Example 18

A chewable tablet prepared by the methods of the present invention was left in the closed trunk of a car. Even when the ambient temperature inside the closed trunk rose to greater than 120° F., the chewable the product was found to have not substantially lost its moisture. The chewable tablet retained much of its short structure, texture, and displayed no stickiness to the wrapper/container. Thus, the chewable tablet maintained its stability and structure under high temperatures.

What is claimed is:

1. A method for forming a chewable composition for administration to a mammal comprising:
a) preparing a nonaqueous phase by applying thermal energy, radiation, or mechanical force to a solid or semisolid nonaqueous composition;
b) preparing an aqueous liquid phase by applying thermal energy, radiation, or mechanical force to an aqueous composition without substantial moisture loss such that the aqueous phase has reduced viscosity compared to said aqueous composition;
c) contacting said nonaqueous phase with said aqueous phase to obtain an emulsion; and
d) optionally adding ingredients selected from the group consisting of: fillers, colorants, humectants, preservatives, taste-masking agents, sweeteners, stabilizers and mixtures thereof; and
e) forming a chewable base material for delivering an active agent.

2. The method of claim 1, further comprising incorporating an active agent into the chewable base material of step (e) at a temperature below that of either step (a) or step (b).

3. The method of claim 1, wherein the nonaqueous phase is prepared at a temperature ranging from about 55° C. to about 120° C.

4. The method of claim 1, wherein the nonaqueous phase is prepared at a temperature ranging from about 55° C. to about 100° C.

5. The method according to claim 1, further comprising adding an emulsifier selected from the group consisting of: acetylated monoglycerides, glycerol esters, lecithin, de-oiled lecithin, enzyme-modified lecithins, purified lecithins, glycerol monostearate, polyglycerol esters, propylene glycol esters, sorbitan esters, polysorbate esters, sodium laurel sulfate, polyethylene glycols, sorbitol mono-, di- and tri-stearates, waxes and mixtures thereof.

6. The method of claim 1, wherein the nonaqueous composition is selected from the group consisting of: chocolate, cocoa butter, palm oil, canola oil, corn oil, sunflower oil, coconut oil, partially hydrogenated soybean oil, partially hydrogenated palm oil, partially hydrogenated coconut oil, partially hydrogenated canola oil, partially hydrogenated cottonseed oil, recinolate, and mixtures thereof.

7. The method of claim 1, wherein the aqueous phase is prepared at a temperature ranging from about 80° F. to about 200° F.

8. The method of claim 1, wherein the aqueous phase is prepared at a temperature ranging from about 80° F. to about 190° F.

9. The method of claim 1, wherein the substantial moisture loss is about 30% or less.

10. The method of claim 1, wherein the substantial moisture loss is about 20% or less.

11. The method of claim 1, wherein the substantial moisture loss is about 10% or less.

12. The method of claim 1, wherein the substantially reduced viscosity represents a reduction in viscosity of from about 2 times to about 40 times.

13. The method of claim 1, wherein the substantially reduced viscosity represents a reduction in viscosity of from about 2 times to about 20 times.

14. The method of claim 1, wherein the substantially reduced viscosity represents a reduction in viscosity of from about 2 times to about 10 times.

15. The method of claim 1, wherein the aqueous composition is selected from the group consisting of: humectants, gums, syrups, or mixtures thereof.

16. The method of claim 1, wherein the aqueous composition is selected from the group consisting of: propylene glycol, starch hydrolysate, isomalt, erythritol, polydextrose, maltitol, lactitol, glycerin, sorbitol, xylitol, mannitol and mixtures thereof.

17. The method of claim 1, wherein the aqueous composition is selected from the group consisting of: gum arabic, gelatin, locust bean gum, guar gum, hydrolyzed guar gum, carrageenan, starches, agar, alginate, pectin and mixtures thereof.

18. The method of claim 1, wherein said active is uncoated, or coated with a taste-masking agent, or an enteric-release coating material or both, or a film-coating material or a mixture thereof.

19. The method of claim 1, wherein said active is a thermally sensitive therapeutic agent.

20. The method of claim 1, wherein said active is selected from the group consisting of: dietary fiber, stannol esters, therapeutically active substances, vitamins, minerals, antacids, cough and cold medications, analgesics, cardiovasular medications, anti-smoking, psycho-therapeutics, antibiotics, and mixtures thereof.

21. The method of claim 1, wherein said forming of step (e) comprises compressing into a tablet, rope-pinching into a tablet, deposition into a specific predetermined geometric shape, or formulating into beads or granules.

22. A chewable product prepared according to the method of claim 1.

23. The method according to claim 1, further comprising adding a bioadhesive agent selected from the group consisting of hydroxypropylmethyl cellulose, ethyl cellulose, acrylic esters, polyvinyl acetates, alcohols and gums.

24. The method according to claim 1, wherein the chewy composition formed in step (e) is stored for a period of time prior to the incorporation of additional ingredients.

25. The method according to claim 24, wherein the period of time is one day.

26. The method according to claim 24, wherein the period of time is about one week.

27. The method according to claim 24, wherein the period of time is about one month.

28. The method according to claim 24, wherein the period of time is about 3 months.

29. The method according to claim 24, wherein the period of time is about 6 months.

30. The method according to claim 24, wherein the period of time is about 1 year.

31. The method according to claim 24, wherein the additional ingredient is selected from an active agent, a flavorant, a colorant, or mixtures thereof.

32. The method according to claim 24, wherein the chewy composition is reheated to a temperature of about 110° F. prior to the incorporation of the additional ingredient.
